(19) United States
(12) Patent Application Publication
(10) Pub. No.: US 2017/0173011 A1
(43) Pub. Date: Jun. 22, 2017

(54) METHODS OF TREATING A BRUTON'S TYROSINE KINASE DISEASE OR DISORDER

(71) Applicant: Celgene Avilomics Research, Inc., Bedford, MA (US)

(72) Inventors: William Frederick Westlin, III, Boxborough, MA (US); Garth Ringheim, Belle Mead, NJ (US)

(21) Appl. No.: 15/124,007

(22) PCT Filed: Mar. 5, 2015
(86) PCT No.: PCT/US15/19056

§ 371 Date: Sep. 6, 2016

Related U.S. Application Data
(60) Provisional application No. 61/949,605, filed on Mar. 7, 2014.

(51) Int. Cl. A61K 31/505; C07D 239/48

(52) U.S. Cl. CPC A61K 31/505; C07D 239/48

(57) ABSTRACT

The present invention provides methods of treating, stabilizing or lessening the severity or progression of a disease or disorder associated with Bruton's tyrosine kinase (BTK) TEC and interleukin-2-inducible T-cell kinase (ITK).

### CROSS REFERENCE TO RELATED APPLICATIONS

The present application claims priority to U.S. provisional application No. 61/949,605, filed Mar. 7, 2014, the entirety of which is hereby incorporated by reference.

### FIELD OF THE INVENTION

The present invention provides methods of treating, stabilizing or lessening the severity or progression of a disease or disorder associated with Bruton's Tyrosine Kinase (“BTK”) TEC and interleukin-2-inducible T-cell kinase (ITK).

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

The present invention provides methods of treating, stabilizing or lessening the severity or progression of one or more diseases and conditions associated with one or more kinases selected from BTK, TEC and interleukin-2-inducible T-cell kinase (ITK). In some aspects, the present invention provides methods of treating, stabilizing or lessening the severity or progression of one or more diseases and conditions associated with one or more kinases selected from BTK, TEC and ITK comprising administering to a patient in need thereof a pharmaceutically acceptable composition comprising N-(3-(5-fluoro-2-(4-(2-methoxyethoxy)phenylamino)pyrimidin-4-ylamino)phenyl)acrylamide (1):

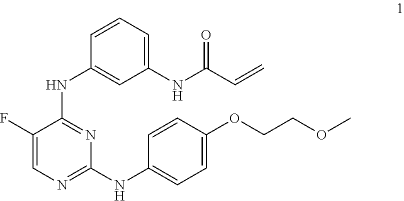 [SMILES: C=CC(=O)Nc1cccc(Nc2nc(Nc3ccc(OCCOC)cc3)ncc2F)c1]

or a pharmaceutically acceptable salt thereof.

In some embodiments, the present invention provides a method of treating, stabilizing or lessening the severity or progression of an autoimmune disorder, wherein the method comprises administering to a patient in need thereof a pharmaceutically acceptable composition comprising Compound 1, or a pharmaceutically acceptable salt thereof. In some embodiments, the autoimmune disorder is selected from lupus (e.g., systemic lupus erythematosis (SLE) or drug-induced lupus erythematosus), Graves' disease, Hashimoto's thyroiditis, myasthenia gravis, mixed connective tissue disease, celiac disease, inflammatory myopathy, diabetes mellitus type 1, and Lambert-Eaton myasthenic syndrome. In some embodiments, the autoimmune disorder is lupus.

In some embodiments, provided methods comprise orally administering to a patient compositions comprising Compound 1, or a pharmaceutically acceptable salt thereof. In some embodiments, such compositions are capsule formulations. In general, provided methods comprise administering a composition which comprises Compound 1, or a pharmaceutically acceptable salt thereof, and one or more pharmaceutically acceptable excipients, such as, for example, binders, diluents, disintegrants, wetting agents, lubricants and adsorbents.

In some embodiments, the present invention also provides dosing regimens and protocols for the administration of Compound 1, or a pharmaceutically acceptable salt thereof, to patients in need thereof.

### BRIEF DESCRIPTION OF THE DRAWINGS

FIG. 1A presents a graph depicting individual autoantibody development in NZBxNZW F1 mice over time.

FIG. 1B presents a graph depicting individual autoantibody development in NZBxNZW F1 mice treated with 50 mg/kg BID Compound 1 besylate.

FIG. 1C presents a graph depicting reduced autoantibody development in NZBxNZW F1 mice treated with 50 mg/kg BID Compound 1 besylate.

FIG. 2 depicts the reduced urine protein levels in NZBxNZW F1 mice treated with Compound 1 besylate.

### DETAILED DESCRIPTION OF THE INVENTION

### Definitions

As used herein, a “disease or disorder associated with one or more kinases selected from BTK, TEC and ITK” means any disease or other deleterious condition in which one or more of BTK, TEC or ITK, or a mutant thereof, is known or suspected to play a role. Accordingly, another embodiment of the present invention relates to preventing, treating, stabilizing or lessening the severity or progression of one or more diseases in which BTK, TEC or ITK, or a mutant thereof, is known or suspected to play a role. Specifically, the present invention relates to a method of treating or lessening the severity of an autoimmune disorder, wherein said method comprises administering to a patient in need thereof Compound 1, or a pharmaceutically acceptable salt thereof, or a pharmaceutically acceptable composition thereof.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with one or more of Bruton's tyrosine kinase (BTK), TEC and interleukin-2-inducible T-cell kinase (ITK).

The term “subject”, as used herein, means a mammal and includes human and animal subjects, such as domestic animals (e.g., horses, dogs, cats, etc.).

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of inventive formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### Compound 1 is an Irreversible BTK Inhibitor

United States published patent application number US 2010/0029610, published Feb. 4, 2010 (“the '610 publication,” the entirety of which is hereby incorporated herein by reference), describes certain 2,4-disubstituted pyrimidine compounds which covalently and irreversibly inhibit activity of one or more protein kinases, including BTK, TEC or ITK. Such compounds include Compound 1, which is designated as compound number I-182 in the '610 publication. The synthesis of Compound 1 is described in detail at Example 20. Compound 1 is active in a variety of assays and therapeutic models demonstrating covalent, irreversible inhibition of BTK (in enzymatic and cellular assays). Accordingly, Compound 1, or a pharmaceutically acceptable salt thereof, is useful for treating one or more disorders associated with activity of one or more kinases selected from BTK, TEC and ITK.

The present invention provides methods of treating, stabilizing or lessening the severity or progression of one or more diseases and conditions associated with one or more kinases selected from BTK, TEC and ITK comprising administering to a patient in need thereof a pharmaceutically acceptable composition comprising Compound 1, or a pharmaceutically acceptable salt thereof, wherein the pharmaceutically acceptable composition is an oral dosage form. In some such embodiments, the pharmaceutically acceptable composition is formulated as a capsule. Such methods, dosing regimens and protocols for the administration of pharmaceutically acceptable compositions comprising Compound 1, or a pharmaceutically acceptable salt thereof, are described in further detail, below.

### I. General Dosing Protocol

As described above, the present invention provides methods of treating, stabilizing or lessening the severity or progression of one or more diseases or conditions associated with one or more kinases selected from BTK, TEC and ITK, wherein the method comprises administering to a patient in need thereof a pharmaceutically acceptable composition comprising Compound 1, or a pharmaceutically acceptable salt thereof. In some embodiments, the present invention provides a method of preventing the progression of a disease or disorder associated with one or more kinases selected from BTK, TEC and ITK. It is understood that although the methods described herein refer to administering Compound 1, such methods are equally applicable to methods of administering a salt form of Compound 1, e.g., a besylate salt of Compound 1. Accordingly, methods provided herein are to be understood to encompass either the administration of Compound 1 or a pharmaceutically acceptable salt thereof.

In some embodiments, provided methods comprise administering to a patient in need thereof a pharmaceutically acceptable composition comprising from about 5% to about 60% of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of Compound 1, based upon total weight of the formulation. In certain embodiments, provided methods comprise administering to a patient in need thereof a pharmaceutically acceptable composition comprising from about 6%, about 7%, about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of Compound 1, based upon total weight of given composition or formulation.

In some embodiments, provided methods comprise administering to a patient in need thereof a pharmaceutically acceptable composition comprising from about 25% to about 75% or about 30% to about 60% or about 40% to about 50% or about 40% to about 45% of Compound 1, or a pharmaceutically acceptable salt thereof, based upon total weight of the formulation. In some embodiments, provided methods comprise administering to a patient in need thereof a pharmaceutically acceptable composition comprising from about 6%, about 7%, about 8%, about 9%, about 10%, about 11%, about 12%, about 13% of Compound 1, or a pharmaceutically acceptable salt thereof, based upon total weight of given composition or formulation. In certain embodiments, provided methods comprise administering to a patient in need thereof a pharmaceutically acceptable composition comprising from about 41%, about 42%, about 43%, about 44% or about 45%, of Compound 1, or a pharmaceutically acceptable salt thereof, based upon total weight of given composition or formulation.

In some embodiments, provided methods comprise administering a pharmaceutically acceptable composition comprising Compound 1 one, two, three, or four times a day. In some embodiments, a pharmaceutically acceptable composition comprising Compound 1 is administered once daily (“QD”). In some embodiments, a pharmaceutically acceptable composition comprising Compound 1 is administered twice daily. In some embodiments, twice daily administration refers to a compound or composition that is administered “BID”. A “BID” dose is a particular dose (e.g., a 125 mg dose) that is administered twice a day (i.e., two doses of 125 mg administered at two different times in one day). In some embodiments, twice daily administration refers to a compound or composition that is administered in two different doses, wherein the first administered dose differs from the second administered dose. For example, a 250 mg dose administered twice daily can be administered as two separate doses, one 150 mg dose and one 100 mg dose, wherein each dose is administered at a different time in one day. Alternatively, a 250 mg dose administered twice daily can be administered 125 mg BID (i.e., two 125 mg doses administered at different times in one day). In some embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, a pharmaceutically acceptable composition comprising Compound 1 is administered three times a day. In some embodiments, a pharmaceutically acceptable composition comprising Compound 1 is administered “TID”, or three equivalent doses administered at three different times in one day. In some embodiments, a pharmaceutically acceptable composition comprising Compound 1 is administered in three different doses, wherein at least one of the administered doses differs from another administered dose. In some embodiments, a pharmaceutically acceptable composition comprising Compound 1 is administered four times a day. In some embodiments, a pharmaceutically acceptable composition comprising Compound 1 is administered “QID”, or four equivalent doses administered at four different times in one day. In some embodiments, a pharmaceutically acceptable composition comprising Compound 1 is administered in four different doses, wherein at least one of the administered doses differs from another administered dose.

In some embodiments, Compound 1 is administered to a patient twice a day, wherein the first administered dose differs from the second administered dose. In some such embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, provided methods comprise administering a pharmaceutically acceptable composition comprising Compound 1 once a day (“QD”). In some embodiments, provided methods comprise administering a pharmaceutically acceptable composition comprising Compound 1 twice a day. In some embodiments, a pharmaceutically acceptable composition comprising Compound 1 is administered once or twice daily fora period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a pharmaceutically acceptable composition comprising Compound 1 is administered once or twice daily for 28 consecutive days (“a 28-day cycle”). In some embodiments, a pharmaceutically acceptable composition comprising Compound 1 is administered once or twice daily for at least one 28-day cycle. In some embodiments, a pharmaceutically acceptable composition comprising Compound 1 is administered once or twice daily for at least two, at least three, at least four, at least five or at least six 28-day cycles. In some embodiments, a pharmaceutically acceptable composition comprising Compound 1 is administered once or twice daily for at least seven, at least eight, at least nine, at least ten, at least eleven or at least twelve 28-day cycles. In some embodiments, a pharmaceutically acceptable composition comprising Compound 1 is administered once or twice daily for at least thirteen, at least fourteen, at least fifteen, at least sixteen, at least seventeen, at least eighteen, at least nineteen or at least twenty 28-day cycles. In some embodiments, a pharmaceutically acceptable composition comprising Compound 1 is administered to a patient for the duration of the patient's life.

In some embodiments, two adjacent 28-day cycles may be separated by a rest period. Such a rest period may be one, two, three, four, five, six, seven or more days during which the patient is not administered a unit dose of Compound 1. In a preferred embodiment, two adjacent 28-day cycles are continuous.

### Unit Dosage Forms

Pharmaceutical compositions for use in the present invention may be prepared as a unit dosage form. A person of ordinary skill will appreciate that the unit dosage forms described herein refer to an amount of Compound 1 as a free base. A person skilled in the art will further appreciate that, when a pharmaceutical composition comprises a salt form of Compound 1, for example a besylate salt form, the amount of the salt form present in the composition is an amount that is equivalent to a unit dose of the free base of Compound 1. For example, a pharmaceutical composition comprising a besylate salt of Compound 1 would contain 34.97 mg of the besylate salt form necessary to deliver an equivalent 25 mg unit dose of the free base of Compound 1.

In some embodiments, provided methods comprise administering to a patient in need thereof a composition comprising a unit dose of Compound 1, wherein the unit dose is about 75 mg to about 750 mg. In some embodiments, provided methods comprise administering to a patient in need thereof a composition comprising a unit dose of Compound 1, wherein the unit dose is about 125 mg to about 750 mg. In some embodiments, provided methods comprise administering to a patient in need thereof a composition comprising a unit dose of Compound 1, wherein the unit dose is about 125 mg to about 500 mg. In some embodiments, provided methods comprise administering to a patient in need thereof a composition comprising a unit dose of Compound 1, wherein the unit dose is about 250 mg to about 500 mg. In some embodiments, a unit dose of Compound 1 is administered once a day (QD). In some embodiments, a unit dose of Compound 1 is administered twice a day . In some embodiments, a unit dose of Compound 1 is administered BID.

In some embodiments, the unit dose of Compound 1 is about 25 mg to 750 mg, or about 25 mg to about 625 mg, or about 25 mg to about 500 mg, or about 25 mg to about 375 mg, or about 25 mg to about 250 mg, or about 25 mg to about 125 mg, or about 25 mg to about 75 mg, or about 75 mg to about 750 mg, or about 75 mg to about 625 mg, or about 75 mg to about 500 mg, or about 75 mg to about 375 mg, or about 75 mg to about 250 mg, or about 75 mg to about 125 mg, or about 125 mg to about 750 mg, or about 125 mg to about 625 mg, or about 125 mg to about 500 mg, or about 125 mg to about 375 mg, or about 125 mg to about 250 mg, or about 250 mg to about 750 mg, or about 250 mg to about 625 mg, or about 250 mg to about 500 mg, or about 250 mg to about 375 mg, or about 375 mg to about 750 mg, or about 375 mg to about 625 mg, or about 375 mg to about 500 mg, or about 500 mg to about 750 mg, or about 500 mg to about 625 mg, or about 625 mg to about 750 mg.

In some embodiments, the unit dose of Compound 1 is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745mg or about 750 mg.

In some embodiments, Compound 1 is administered two, three or four times a day, wherein each dose is identical. In some embodiments, Compound 1 is administered two, three or four times a day, wherein at least one dose is different from another dose. In some such embodiments, each dose may be independently selected from those doses or dose ranges in the two preceeding paragraphs.

In some embodiments, provided methods comprise administering to a patient in need thereof a pharmaceutical composition comprising a unit dose of Compound 1. In some such embodiments, the unit dose is about 25 mg, about 50 mg, about 75 mg, about 100 mg, about 125 mg, about 150 mg, about 175 mg, about 200 mg, about 225 mg or about 250 mg.

### II. Uses of Compounds And Pharmaceutically Acceptable Compositions

Compound 1 and compositions described herein are generally useful for the inhibition of protein kinase activity of one or more enzymes. Examples of kinases that are inhibited by Compound 1 and compositions described herein and against which the methods described herein are useful include BTK and other TEC-kinases, including ITK, TEC, BMX and RLK, or a mutant thereof.

Bruton's tyrosine kinase (Btk) is a member of the Tec family of cytosolic tyrosine kinases and is expressed exclusively in B cells and cells of the myeloid lineage. Btk has a well characterized essential role in B cells that is highlighted by the human primary immune deficiency disease, X-linked agammaglobulinemia (XLA), which results from mutation in the Btk gene and produces a functionally inactive protein (Smith et al., “X-linked agammaglobulinemia: lack of mature B lineage cells caused by mutations in the Btk kinase,” Springer Semin. Immunopathol. 1998, 19:369-381). XLA patients display a B cell differentiation block at the pro-B to pre-B cell transition (Campana et al., “Phenotypic features and proliferative activity of B cell progenitors in X-linked agammaglobulinemia,” J. Immunol. 1990, 145:1675-1680). As a result of incomplete B cell differentiation, these patients have a near complete absence of mature B cells in the peripheral blood (Campana et al., “Phenotypic features and proliferative activity of B cell progenitors in X-linked agammaglobulinemia,” J. Immunol. 1990, 145:1675-1680) and cannot produce immunoglobulins of any class (Conley, “B cells in patients with X-linked agammaglobulinemia,” J. Immunol. 1985, 134:3070-3074; Nonoyama et al., “Functional analysis of peripheral blood B cells in patients with X-linked agammaglobulinemia,” J. Immunol. 1998, 161:3925-3929). Furthermore, the human XLA phenotype is recapitulated, although less severely, in Btk knock-out mice (Khan et al., “Defective B cell development and function in Btk-deficient mice,” Immunity. 1995, 3:283-299) and in xid mice which have a naturally occurring Btk mutation (Rawlings et al., “Mutation of unique region of Bruton's tyrosine kinase in immunodeficient XID mice,” Science 1993, 261:358-361). These Btk deficient mice display a 50% reduction in circulating B-2 cells, an absence of CD5+ B-1 cells and a failure to respond to T cell independent type II antigens (Rawlings et al., “Mutation of unique region of Bruton's tyrosine kinase in immunodeficient XID mice,” Science 1993, 261:358-361; Wicker et al., “X-linked immune deficiency (xid) of CBA/N mice,” Curr. Top. Microbiol. Immunol. 1986, 124:87-101; Sideras et al., “Molecular and cellular aspects of X-linked agammaglobulinemia,” Adv. Immunol. 1995, 59:135-223; Satterthwaite et al., “Btk dosage determines sensitivity to B cell antigen receptor cross-linking,” Proc. Natl. Acad. Sci. U.S.A 1997, 94:13152-13157) demonstrating a requirement for Btk in normal B cell development and function.

Specifically, Btk plays an essential role in the B cell receptor (BCR) signaling pathway. Antigen binding of the BCR results in B cell receptor oligomerization, leading to interaction of Syk and Lyn kinases with aggregated immunoreceptor tyrosine-based activation motifs (ITAMS) on the CD79 subunit of the BCR and subsequent phosphorylation and activation (Gauld et al., “B cell antigen receptor signaling: roles in cell development and disease,” Science 2002, 296:1641-1642). Lyn and Syk phosphorylate Btk on Tyr551 resulting in activation of the kinase (Rawlings et al., “Activation of BTK by a phosphorylation mechanism initiated by SRC family kinases,” Science 1996, 271:822-825; Park et al., “Regulation of Btk function by a major autophosphorylation site within the SH3 domain,” Immunity 1996, 4:515-525; Baba et al., “BLNK mediates Syk-dependent Btk activation,” Proc. Natl. Acad. Sci. U.S.A 2001, 98:2582-2586). Once activated, Btk translocates to the lipid membrane where it forms a signaling complex with proteins such as Blnk, Lyn, and Syk and phosphorylates PLCγ2 (Baba et al., “BLNK mediates Syk-dependent Btk activation,” Proc. Natl. Acad. Sci. U.S.A 2001, 98:2582-2586; Tsukada et al., “Btk and BLNK in B cell development. Adv. Immunol. 2001, 77:123-162). This in turn leads to downstream release of intracellular Ca²⁺ stores and propagation of the BCR signaling pathway through Erk and NF-κB signaling that ultimately results in transcriptional changes to foster B cell survival, proliferation, and/or differentiation (Baba et al., “BLNK mediates Syk-dependent Btk activation,” Proc. Natl. Acad. Sci. U.S.A 2001, 98:2582-2586; Maas et al., “Role of Bruton's tyrosine kinase in B cell development,” Dev. Immunol. 2001, 8:171-181; Mohamed et al., “Bruton's tyrosine kinase (Btk): function, regulation, and transformation with special emphasis on the PH domain,” Immunol. Rev. 2009, 228:58-73).

While essential in the normal development and function of B cells, there are several pathologies that have been attributed in part to dysregulated BCR activity. The expression of Btk is highly restricted to cells of hematopoietic lineage including B lymphocytes, mast cells, monocytes, and osteoclasts. This highly restricted expression pattern of Btk together with the prominent role of Btk in the BCR signaling pathway makes it an attractive drug target for the treatment of B cell-associated autoimmune diseases. These include diseases of autoreactivity such as that observed in lupus, multiple sclerosis (MS), and rheumatoid arthritis (RA) in which B cells inappropriately break self-tolerance to produce autoantibodies and contribute to autoimmune disease (Edwards et al., “B-cell targeting in rheumatoid arthritis and other autoimmune diseases,” Nat. Rev. Immunol. 2006, 6:394-403; Teng et al., “Targeted therapies in rheumatoid arthritis: Focus on rituximab,” Biologics 2007, 1:325-333; Edwards et al., “Prospects for B-cell-targeted therapy in autoimmune disease,” Rheumatology (Oxford) 2005, 44:151-156). It has also been recently recognized that BCR signaling contributes to several B cell malignancies such as chronic lymphocytic leukemia (CLL) (Chen et al., “ZAP-70 directly enhances IgM signaling in chronic lymphocytic leukemia,” Blood 2005, 105:2036-2041; Hoellenriegel et al., “The phosphoinositide 3′-kinase delta inhibitor, CAL-101, inhibits B-cell receptor signaling and chemokine networks in chronic lymphocytic leukemia,” Blood 2011, 118:3603-3612; Stevenson et al., “B-cell receptor signaling in chronic lymphocytic leukemia,” Blood 2011, 118:4313-4320), mantle cell leukemia (MCL) and subsets of diffuse large B cell lymphoma (DLBCL) (Suljagic et al., “The Syk inhibitor fostamatinib disodium (R788) inhibits tumor growth in the Emu-TCL1 transgenic mouse model of CLL by blocking antigen-dependent B-cell receptor signaling,” Blood 2010, 116:4894-4905; Chen et al., “SYK-dependent tonic B-cell receptor signaling is a rational treatment target in diffuse large B-cell lymphoma,” Blood 2008, 111:2230-2237; Davis et al., “Chronic active B-cell-receptor signalling in diffuse large B-cell lymphoma,” Nature 2010, 463:88-92; Lenz et al., “Molecular subtypes of diffuse large B-cell lymphoma arise by distinct genetic pathways,” Proc. Natl. Acad. Sci. U.S.A 2008, 105:13520-13525; Pighi et al., “Phospho-proteomic analysis of mantle cell lymphoma cells suggests a pro-survival role of B-cell receptor signaling,” Cell Oncol. (Dordr.) 2011, 34:141-153; Baran-Marszak et al., “Constitutive and B-cell receptor-induced activation of STAT3 are important signaling pathways targeted by bortezomib in leukemic mantle cell lymphoma,” Haematologica 2010, 95:1865-1872). However, until recently, therapies that target the B cell have resulted in depletion of the B cell repertoire while therapeutic strategies that reduce BCR activity are relatively new for treatment of these diseases. Promising recent clinical data generated by inhibition of distinct BCR signaling components, including Syk and PI3Kδ with fostamatinib and GS-1101(CAL-101), respectively, have emerged providing great excitement for this approach. Inhibition of Syk with fostamatinib has demonstrated efficacy in preclinical models of inflammatory disease and in human clinical trials in autoimmune diseases (RA and ITP) as well as in B cell malignancies dependent on BCR signaling such as CLL (Chen et al., “SYK-dependent tonic B-cell receptor signaling is a rational treatment target in diffuse large B-cell lymphoma,” Blood 2008, 111:2230-2237; Friedberg et al., “Inhibition of Syk with fostamatinib disodium has significant clinical activity in non-Hodgkin lymphoma and chronic lymphocytic leukemia,” Blood 2010, 115:2578-2585; Genovese et al., “An oral Syk kinase inhibitor in the treatment of rheumatoid arthritis: A three-month randomized, placebo-controlled, phase II study in patients with active rheumatoid arthritis that did not respond to biologic agent,” Arthritis Rheum. 2011, 63:337-345; Podolanczuk et al., “Of mice and men: an open-label pilot study for treatment of immune thrombocytopenic purpura by an inhibitor of Syk,” Blood 2009, 113:3154-3160; Braselmann et al., “R406, an orally available spleen tyrosine kinase inhibitor blocks fc receptor signaling and reduces immune complex-mediated inflammation,” J. Pharmacol. Exp. Ther. 2006, 319:998-1008).

Similarly, inhibition of PI3Kδ with GS-1101 has also shown promising results in CLL (Hoellenriegel et al., “The phosphoinositide 3′-kinase delta inhibitor, CAL-101, inhibits B-cell receptor signaling and chemokine networks in chronic lymphocytic leukemia,” Blood 2011, 118:3603-3612; Lannutti et al., “CAL-101, a p110delta selective phosphatidylinositol-3-kinase inhibitor for the treatment of B-cell malignancies, inhibits PI3K signaling and cellular viability,” Blood 2011, 117:591-594; Herman et al., “Phosphatidylinositol 3-kinase-delta inhibitor CAL-101 shows promising preclinical activity in chronic lymphocytic leukemia by antagonizing intrinsic and extrinsic cellular survival signals,” Blood 2010, 116:2078-2088). Btk, downstream of Syk and PI3Kδ in the BCR signaling pathway, also represents an attractive drug target in diseases characterized by aberrant B cell activity. Moreover, owing to its highly restricted expression pattern in B cells and myeloid cells, Btk provides an opportunity for selective therapeutic targeting. Preclinically, small molecule inhibition of Btk with CGI1746 and PCI-32765 demonstrated therapeutic activity in several models of autoimmune disease (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080; Chang et al., “The Bruton tyrosine kinase inhibitor PCI-32765 ameliorates autoimmune arthritis by inhibition of multiple effector cells,” Arthritis Res. Ther. 2011, 13:R115; Di Paolo et al., “Specific Btk inhibition suppresses B cell- and myeloid cell-mediated arthritis,” Nat. Chem. Biol. 2011, 7:41-50). PCI-32765 has demonstrated initial anti-tumor activity against B cell lymphomas in canines (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080) and is showing promising results in early clinical development for the treatment of B cell malignancies (Harrison, “Trial watch: BTK inhibitor shows positive results in B cell malignancies,” Nat. Rev. Drug Discov. 2012, 11:96), providing evidence that Btk represents a viable and efficacious therapeutic target.

Recently, articles describing the pre-clinical efficacy in mouse models of autoimmune disorders in common with SLE have been described. Cyclophophamide and CTLA4Ig have been shown to inhibit lupus nephritis in NZBxNZW F1 mice (see Schiffer et al., 2003, J Immunol 171: 489-497; Daikh and Wofsy, J Imunol 2001;166:2913-2916). Similarly, inhibition of lupus symptoms in NZBxNZW F1 mice has been described for a topoisomerase I inhibitor (Frese-Schaper et al., J Immunol 2010; 184:2175-2182). A Pfizer compound (PF-06250112) with Btk inhibition activity has been reported to be efficacious in a mouse NZBxNZW F1 SLE model, demonstrating reduced spontaneous germinal center formation, plasma cells, anti dsDNA, proteinurea, and Ig deposits. (Rankin et al., J Immunol 2013; 191(9):4540-50). A compound reported by Roche (RN486) was reported to be efficacious in a mouse NZBxNZW F1 mouse model, showing reduced plasma cells, anti dsDNA IgG (not IgM) deposits. Min-Osorio et al., Arthritis Rheum. 2013 September; 65(9):2380-91. A Btk inhibitor reported by Pharmacyclics (PCI 32765/Ibrutinib) was shown to be efficacious in a B6.sle1 mouse SLE model, and demonstrated reduced anti histone, anti ssDNA, kidney lymphocytes and renal damage in these animals (Hutcheson et al., Arthritis Res & Therapy 2012, 14:R243).

Without wishing to be bound by theory, it is believed that attenuation of B-cell and T-cell activation via inhibition of one or more of BTK, TEC or ITK can modulate autoimmune pathologies while avoiding general immunosuppression observed with many current treatments. Inhibition of B-cell and T-cell functions implicated in SLE pathology is thought to provide a therapeutically beneficial reduction in pro-inflammatory activities, including reduced expansion of autoreactive immune cells, trafficking of immune cells to affected tissue areas of pathology, localizing of activated immune cells to disease propagating lymphoid organs, and production of pro-inflammatory cytokine and chemokine mediators. Accordingly, the present invention provides methods of treating, stabilizing or lessening the severity or progression of an autoimmune disorder comprising inhibiting the activity of one or more kinases selected from BTK, TEC or ITK.

### Lupus

Lupus erythematosus is a name given to a collection of autoimmune diseases in which the human immune system becomes hyperactive and attacks normal, healthy tissues. Inflammation caused by lupus can affect many different body systems, including joints, skin, kidneys, blood cells, heart, and lungs.

Lupus can be difficult to diagnose because its signs and symptoms often mimic those of other ailments. The most distinctive sign of lupus, a facial rash that resembles the wings of a butterfly unfolding across both cheeks, occurs in many but not all cases of lupus.

Lupus erythematosus may manifest as a systemic disease or in a purely cutaneous form also known as incomplete lupus erythematosus. Lupus has four main types: systemic, discoid, drug-induced and neonatal. Of these, systemic lupus erythematosus is the most common and serious form.

An estimated 5 million people worldwide have some form of lupus. 70% of cases diagnosed are systemic lupus erythematosus, and females are 6-10 times more likely than males to be affected. 20% of people with lupus will have a parent or sibling who already has or may develop lupus and about 5% of children born to individuals with lupus will eventually develop lupus. In the United States, the peak occurrence of lupus is between ages 15 and 40.

Current treatments for lupus are primarily designed to manage symptoms rather than alter disease state. These include avoiding sun exposure, rest, NSAIDs and corticosteroids. Current treatments for skin manifestations (cutaneous lupus erythematosis; CLE) include the use of topical corticosteroids, antimalarials such as plaquenil (hydroxychloroquine), and immunosuppressants that are either of limited effect or present significant safety concerns that limit their use. Plaquenil is often used to treat the arthritic type symptoms associated with joint damage and to reduce the risk of long term disability.

Rituxan® (rituximab), an antibody immunosuppressant, is sometimes used off-label for moderate to severe patients with mixed results. Benlysta is an antibody therapeutic which inhibits B-cell activating factor (BAFF) protein activity implicated in SLE pathology with limited success.

### Role of B Cells in Lupus

The role of B cells in the pathogenesis of murine lupus has been recently investigated. It is generally accepted that B cells may play pathogenic roles not only through conventional autoantibody-mediated mechanisms, but also by performing antibody-independent regulatory functions. Whether B cells also play antibody-independent roles in human SLE remains to be determined. Recently, scientists have investigated the possibility of B cell depletion as a treatment for SLE.

Rituximab is a chimeric mouse/human monoclonal antibody directed against the B cell specific antigen CD20, an integral membrane protein believed to function in B cell cycle initiation and differentiation. Cell surface expression of CD20 begins at the early pre-B cell stage and is maintained throughout mature B cell development. It decreases substantially in early plasmablasts and is extinguished upon terminal differentiation into mature plasma cells. Owing to its high and relatively sustained expression on neoplastic and normal B cells, CD20 represents an ideal target for immunotherapy of an ever-growing variety of B cell disorders, both malignant and nonmalignant. Indeed, since its approval in 1997 for the treatment of non-Hodgkin's lymphoma, the use of rituximab has expanded into additional malignant conditions as well as autoimmune diseases of proven or presumed B cell origin. Such applications have been predicated on the basis of the ability of rituximab to profoundly deplete nonmalignant B cells for prolonged periods of time both in patients with lymphoma and patients with autoimmune diseases. However, the actual immunologic and clinical effects of rituximab in autoimmune diseases remain to be formally explored.

Rituximab has been studied in clinical trials for the treatment of SLE. Looney et al., Arthritis & Rheumatism 2004, 50(8), 2580-2589; Leandro et al., Rheumatology 2005, 44, 1542-1545. In a dose escalation phase I/II study, rituximab-induced B cell depletion translated into a significant improvement in SLE disease activity even in the absence of substantial serologic responses. This observation is consistent with the autoantibody-independent role of B cells in SLE, which has been demonstrated in murine studies.

Further, SLE patients treated with rituximab, intravenous cyclophosphamide and corticosteroids exhibited clinical improvement and a lowering of double-stranded DNA (dsDNA) antibody levels. Rituximab resulted in substantial depletion of B cells in the peripheral blood (>99%); the overwhelming majority of patients in whom this depletion was achieved improved clinically, with some going into remission.

The highly restricted expression pattern of Bruton's tyrosine kinase (Btk), together with the prominent role of Btk in the B cell receptor signaling pathway, makes Btk an attractive drug target for treatment of complex autoimmune disorders like lupus with Compound 1.

Compound 1 is a potent, selective, orally administered small molecule inhibitor of Btk, which is an integral component of the B cell receptor signaling complex with distribution limited primarily to B lymphocytes and myeloid cells. Btk plays a crucial role in B cell development and function. Compound 1 inhibits Btk activity by binding with high affinity to the adenosine triphosphate (ATP) binding site of Btk. Compound 1 forms a covalent bond with the target Btk protein, providing rapid, complete, and prolonged/irreversible inhibition of Btk activity, both in vitro and in vivo.

Due to its covalent inhibitory mechanism of action, Compound 1 can be administered at therapeutically beneficial levels with long lasting effects, thus improving the therapeutic utility of the compound while providing a safety margin of the drug relative to alternative treatments.

In some embodiments, the present invention provides a method of treating, stabilizing or lessening the severity or progression of a disorder mediated by one or more kinases selected from BTK, TEC and ITK comprising the step of administering to a patient in need thereof N-(3-(5-fluoro-2-(4-(2-methoxyethoxy)phenylamino)pyrimidin-4-ylamino)phenyl)acrylamide, or a pharmaceutically acceptable salt thereof. In some embodiments, provided methods comprise administering to a patient in need thereof a pharmaceutically acceptable composition comprising N-(3-(5-fluoro-2-(4-(2-methoxyethoxy)phenylamino)pyrimidin-4-ylamino)phenyl)acrylamide, or a pharmaceutically acceptable salt thereof.

As used herein, the term “disorder mediated by one or more kinases selected from BTK, TEC and ITK” disorders or conditions as used herein means any disease or other deleterious condition in which one or more kinases selected from BTK, TEC and ITK, or a mutant thereof, is known or suspected to play a role. Accordingly, another embodiment of the present invention relates to treating, stabilizing or lessening the severity or progression of one or more diseases in which one or more kinases selected from BTK, TEC and ITK, or a mutant thereof, is known or suspected to play a role. Specifically, the present invention relates to a method of treating, stabilizing or lessening the severity or progression of an autoimmune disorder, wherein said method comprises administering to a patient in need thereof Compound 1, or a pharmeceutically acceoptable salt thereof, or a composition according to the present invention. In some embodiments, the autoimmune disorder or condition is selected from lupus (e.g., systemic lupus erythematosis (SLE) or drug-induced lupus erythematosus), Graves' disease, Hashimoto's thyroiditis, myasthenia gravis, mixed connective tissue disease, celiac disease, inflammatory myopathy, diabetes mellitus type 1, and Lambert-Eaton myasthenic syndrome. In some embodiments, lupus is not lupus nephritis.

III. Methods of Treating Diseases or Disorders Associated with BTK

In some embodiments, the present invention provides a method of treating, stabilizing or lessening the severity or progression of an autoimmune disorder, the method comprising administering to a patient in need thereof a therapeutically effective amount of a pharmaceutically acceptable composition comprising Compound 1, or a pharmaceutically acceptable salt thereof, wherein the pharmaceutically acceptable composition is administered as an oral dosage form. In some such embodiments, the oral dosage form is a capsule.

In some embodiments, the present invention provides a method of treating, stabilizing or lessening the severity or progression of an autoimmune disorder, for example lupus, the method comprising administering to a patient in need thereof a solid oral dosage form comprising a unit dose of Compound 1, wherein the unit dose is about 25 mg, about 50 mg, about 75 mg, about 100 mg, about 125 mg, about 150 mg, about 175 mg, about 200 mg, about 225 mg or about 250 mg.

In some embodiments, the present invention provides a method of treating, stabilizing or lessening the severity or progression of an autoimmune disorder, for example, lupus, the method comprising administering to a patient in need thereof a pharmaceutical composition comprising Compound 1.

In some embodiments, the present invention provides a method of treating, stabilizing or lessening the severity or progression of an autoimmune disorder, for example, lupus, the method comprising administering to a patient in need thereof a pharmaceutical composition comprising about 25 mg to about 750 mg, or about 25 mg to about 625 mg, or about 25 mg to about 500 mg, or about 25 mg to about 375 mg, or about 25 mg to about 250 mg, or about 25 mg to about 125 mg, or about 25 mg to about 75 mg, or about 75 mg to about 750 mg, or about 75 mg to about 625 mg, or about 75 mg to about 500 mg, or about 75 mg to about 375 mg, or about 75 mg to about 250 mg, or about 75 mg to about 125 mg, or about 125 mg to about 750 mg, or about 125 mg to about 625 mg, or about 125 mg to about 500 mg, or about 125 mg to about 375 mg, or about 125 mg to about 250 mg, or about 250 mg to about 750 mg, or about 250 mg to about 625 mg, or about 250 mg to about 500 mg, or about 250 mg to about 375 mg, or about 375 mg to about 750 mg, or about 375 mg to about 625 mg, or about 375 mg to about 500 mg, or about 500 mg to about 750 mg, or about 500 mg to about 625 mg, or about 625 mg to about 750 mg of Compound 1.

In some embodiments, the present invention provides a method of treating, stabilizing or lessening the severity or progression of an autoimmune disorder, for example, lupus, the method comprising administering to a patient in need thereof a therapeutically effective amount of Compound 1, wherein the therapeutically effective amount is a total daily dose selected from about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a total daily dose of Compound 1 is administered as a single dose. In some embodiments, a total daily dose of Compound 1 is administered as two, three or four doses in one day, wherein each dose is identical. In some embodiments, a total daily dose of Compound 1 is administered as two, three or four doses in one day, wherein at least one dose is different from another dose. When more than one dose is administered in one day, the doses are independently selected from about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a pharmaceutically acceptable composition comprising Compound 1 is administered twice daily. In some embodiments, a pharmaceutically acceptable composition comprising Compound 1 is administered “BID”. In some embodiments, a pharmaceutically acceptable composition comprising Compound 1 is administered in two different doses, wherein the first administered dose differs from the second administered dose.

In some embodiments, a pharmaceutically acceptable composition comprising Compound 1 is administered three times a day. In some embodiments, a pharmaceutically acceptable composition comprising Compound 1 is administered “TID”. In some embodiments, a pharmaceutically acceptable composition comprising Compound 1 is administered in three different doses, wherein at least one of the administered doses differs from another administered dose. In some embodiments, a pharmaceutically acceptable composition comprising Compound 1 is administered four times a day. In some embodiments, a pharmaceutically acceptable composition comprising Compound 1 is administered “QID”. In some embodiments, a pharmaceutically acceptable composition comprising Compound 1 is administered in four different doses, wherein at least one of the administered doses differs from another administered dose.

In some embodiments, a total daily dose of Compound 1 is administered once daily (QD), wherein the dose is selected from 75 mg, 100 mg, 125 mg, 250 mg, 375 mg, 500 mg, 625 mg or 750 mg. In some embodiments, Compound 1 is administered 125 mg QD. In some embodiments, a total daily dose of Compound 1 is administered twice daily, wherein each dose is independently selected from 75 mg, 100 mg, 125 mg or 250 mg. In some embodiments, Compound 1 is administered 125 mg BID. In some embodiments, Compound 1 is administered 250 mg BID.

In some embodiments, provided methods comprise administering to a patient a total daily dose of 125 mg. In some embodiments, provided methods comprise administering to a patient a total daily dose of 250 mg. In some embodiments, provided methods comprise administering to a patient a total daily dose of 375 mg. In some embodiments, provided methods comprise administering to a patient a total daily dose of 500 mg.

In some embodiments, a total daily dose of 375 mg of Compound 1 is administered to a patient twice a day, wherein the first administered dose differs from the second administered dose. In some such embodiments, a total daily dose of 375 mg of Compound 1 is administered as one 250 mg dose and one 125 mg dose.

In some embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, a total daily dose of 375 mg of Compound 1 is administered according to the following dosing schedule:

(i) about 250 mg administered in the morning; and

(ii) about 125 mg administered in the evening.

In some such embodiments, the two doses are administered at least 4 hours apart. In some embodiments, the two doses are administered at least 8 hours apart. In some embodiments, the two doses are administered at least 12 hours apart. In some such embodiments, one dose (for example, 250 mg) is administered in the morning and the second dose (for example, 125 mg) is administered in the evening.

In some embodiments, a therapeutically effective amount of Compound 1 is administered over a period of 28 consecutive days (“a 28-day cycle”). In some embodiments, a therapeutically effective amount of Compound 1 is administered for two, three, four, five or six 28-day cycles. In some embodiments, a therapeutically effective amount of Compound 1 is administered for seven, eight, nine, ten, eleven, twelve or more 28-day cycles. In some embodiments, a pharmaceutically acceptable composition comprising Compound 1 is administered for at least thirteen, at least fourteen, at least fifteen, at least sixteen, at least seventeen, at least eighteen, at least nineteen or at least twenty 28-day cycles. In some embodiments, a therapeutically effective amount of Compound 1 is administered to a patient for the duration of the patient's life.

In some embodiments, two adjacent 28-day cycles may be separated by a rest period. Such a rest period may be one, two, three, four, five, six, seven or more days during which the patient is not administered a unit dose of Compound 1. In a preferred embodiment, two adjacent 28-day cycles are continuous.

In some embodiments, the total daily dose is selected from about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a total daily dose of Compound 1 is administered as a single dose. In some embodiments, a total daily dose of Compound 1 is administered as two, three or four doses in one day, wherein each dose is identical. In some embodiments, a total daily dose of Compound 1 is administered as two, three or four doses in one day, wherein at least one dose is different from another dose. When more than one dose is administered in one day, the doses are independently selected from about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some such embodiments, a total daily dose of 375 mg of Compound 1 is administered to a patient twice a day, wherein the first administered dose differs from the second administered dose. In some embodiments, a total daily dose of 375 mg of Compound 1 comprises a 250 mg dose and a 125 mg dose, wherein each of the 250 mg dose and the 125 mg dose are administered at different times during one day.

In some embodiments, provided methods comprise administering Compound 1, or a pharmaceutically acceptable salt thereof, to a population consisting primarily of female subjects. In some embodiments, provided methods comprise administering Compound 1, or a pharmaceutically acceptable salt thereof, to a population consisting primarily of male subjects. In some embodiments, provided methods comprise administering Compound 1, or a pharmaceutically acceptable salt thereof, to a population consisting of both male and female subjects.

### IV. Formulations Comprising Compound 1

As described above, provided methods comprise administering to a patient in need thereof a pharmaceutically acceptable composition comprising Compound 1, wherein the pharmaceutically acceptable composition is an oral dosage form. In some embodiments, the pharmaceutically acceptable composition is formulated as a capsule.

In certain embodiments, provided methods comprise administering to a patient in need thereof a pharmaceutically acceptable composition which comprises Compound 1, and one or more pharmaceutically acceptable excipients, such as, for example, binders, film coatings, diluents, disintegrants, wetting agents, lubricants and adsorbents, or combinations thereof. One skilled in the art will readily appreciate that the category under which a particular component is listed is not intended to be limiting; in some cases a particular component might appropriately fit in more than one category. Also, as will be appreciated, the same component can sometimes perform different functions, or can perform more than one function, in the context of a particular formulation, for example depending upon the amount of the ingredient and/or the presence of other ingredients and/or active compound(s). In some embodiments, the pharmaceutically acceptable composition is a blended powder.

i. Binders and Diluents

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel®), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

ii. Disintegrants

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

iii. Wetting Agents

Wetting agents, also referred to as bioavailability enhancers, are well known in the art and typically facilitate drug release and absorption by enhancing the solubility of poorly-soluble drugs. Representative wetting agents include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the wetting agent is a poloxamer. In some such embodiments, the poloxamer is poloxamer 407. In some embodiments, compositions for use in the present invention comprise from about 1% to about 30% by weight of wetting agent, based upon total weight of the blended powder.

iv. Lubricants

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

v. Adsorbents

Pharmaceutical compositions of the present invention may further comprise one or more adsorbents. Representative adsorbents include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% adsorbent, based upon total weight of given formulation.

vi. N-(3-(5-fluoro-2-(4-(2-methoxyethoxy)phenylamino)pyrimidin-4-ylamino)phenyl)acrylamide

As described above, the present invention provides a method of treating an autoimmune disorder, the method comprising administering to a patient in need thereof Compound 1. In some embodiments, provided methods comprise administering a pharmaceutically acceptable composition comprising Compound 1. Thus, in some embodiments, provided methods comprise administering to a patient in need thereof a besylate salt of Compound 1.

In some embodiments, provided methods comprise administering to a patient in need thereof a pharmaceutically acceptable composition comprising from about 5% to about 60% of Compound 1, based upon total weight of the formulation. In some embodiments, provided methods comprise administering to a patient in need thereof a pharmaceutically acceptable composition comprising from about 5% to about 60% of the besylate salt of Compound 1, based upon total weight of the formulation. In some embodiments, provided methods comprise administering to a patient in need thereof a pharmaceutically acceptable composition comprising from about 5% to about 15% or about 5% to about 10% or about 7% to about 8% of Compound 1, based upon total weight of the composition. In some embodiments, provided methods comprise administering to a patient in need thereof a pharmaceutically acceptable composition comprising from about 5% to about 15% or about 7% to about 15% or about 7% to about 10% or about 9% to about 12% of the besylate salt of Compound 1, based upon total weight of the composition. In some embodiments, provided methods comprise administering to a patient in need thereof a pharmaceutically acceptable composition comprising from about 25% to about 50% or about 25% to about 35% or about 30% to about 35% of Compound 1, based upon total weight of the formulation. In some embodiments, provided methods comprise administering to a patient in need thereof a pharmaceutically acceptable composition comprising from about 25% to about 75% or about 30% to about 60% or about 40% to about 50% or about 40% to about 45% of the besylate salt of Compound 1, based upon total weight of the formulation. In certain embodiments, provided methods comprise administering to a patient in need thereof a pharmaceutically acceptable composition comprising from about 7%, about 8%, about 9%, about 10%, about 11%, about 12%, about 13%, about 25%, about 28%, about 29%, about 30%, about 31%, about 32%, about 33%, about 34%, about 35%, about 36% or about 37% of Compound 1, based upon total weight of given composition or formulation. In certain embodiments, provided methods comprise administering to a patient in need thereof a pharmaceutically acceptable composition comprising from about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of the besylate salt of Compound 1, based upon total weight of given composition or formulation.

In some such embodiments, provided methods comprise administering to a patient in need thereof a pharmaceutical composition comprising a unit dose of Compound 1. In some such embodiments, provided methods comprise administering to a patient in need thereof a pharmaceutical composition comprising a unit dose of Compound 1, wherein Compound 1 is in the form of a besylate salt. In some such embodiments, the unit dose is an amount sufficient to provide about 25 mg, about 50 mg, about 75 mg, about 100 mg, about 125 mg, about 150 mg, about 175 mg, about 200 mg, about 225 mg or about 250 mg of the free base of Compound 1. In some embodiments, the pharmaceutical composition comprising Compound 1, or a besylate salt thereof, is a solid oral dosage form.

In some embodiments, the present invention provides a method of treating, stabilizing or lessening the severity or progression of an autoimmune disorder, wherein said method comprises administering to a patient in need thereof Compound 1, or a besylate salt thereof, or a pharmaceutically acceptable composition thereof

In some embodiments, the present invention provides a method of treating, stabilizing or lessening the severity or progression of lupus, wherein said method comprises administering to a patient in need thereof Compound 1, or a besylate salt or a pharmaceutically acceptable composition thereof.

In some embodiments, the present invention provides a method of treating, stabilizing or lessening the severity or progression of an autoimmune disorder, for example, lupus, the method comprising administering to a patient in need thereof a pharmaceutical composition comprising the besylate salt of Compound 1, wherein the amount of besylate salt is sufficient to deliver about 75 mg, about 100 mg, about 125 mg, about 250 mg, about 375 mg, about 500 mg, about 625 mg or about 750 mg of the free base of Compound 1. In some such embodiments, the pharmaceutical composition further comprises one or more pharmaceutically acceptable excipients selected from binders, film coating, diluents, disintegrants, wetting agents, lubricants and adsorbents. In some such embodiments, the pharmaceutical composition comprises one or more pharmaceutically acceptable excipients selected from microcrystalline cellulose, lactose monohydrate, sodium starch, poloxamer 407, fumed silica and magnesium stearate. In some embodiments, the pharmaceutical composition is selected from those in Table 1:

TABLE 1
Pharmaceutical Formulations Comprising Compound 1

| | Amount per | | Amount per | |
|---|---|---|---|---|
| Component | 25 mg Capsule | | 125 mg Capsule | |
| Capsule shell | 1, size 0 | | 1, size 0 | |
| | white capsule | | white capsule | |
| N-(3-(5-fluoro-2-(4-(2- | 34.97 mg | | 174.26 mg | |
| methoxyethoxy)phenylamino)- | (25 mg free base) | | (125 mg free base) | |
| pyrimidin-4- | | | | |
| ylamino)phenyl)acrylamide | | | | |
| besylate (API) | | | | |
| Microcrystalline cellulose | 186.03 | mg | 101.72 | mg |
| Lactose monohydrate | 32.50 | mg | 41.50 | mg |
| Sodium starch glycolate | 32.50 | mg | 41.50 | mg |
| Poloxamer 407 | 32.50 | mg | 41.50 | mg |
| Fumed silica | 3.25 | mg | 4.15 | mg |
| Magnesium stearate | 3.25 | mg^{†} | 10.38 | mg^{‡} |

^{†}0.5% (1.625 mg) intragranular; 0.5% (1.625 mg) extragranular.
^{‡}2.0% (8.30 mg) intragranular; 0.5% (2.08 mg) extragranular.

### V. Process for Preparing Pharmaceutical Compositions

Dry Blend Process:

Milled N-(3 -(5 -fluoro-2-(4-(2-methoxyethoxy)phenylamino)pyrimidin-4-ylamino)phenyl)acrylamide besylate, milled microcrystalline cellulose, milled sodium starch glycolate, milled lactose monohydrate, milled poloxamer 407, and sieved fumed silica are weighed and mechanically blended. An intragranular portion of sieved magnesium stearate (0.5% or 2.0%, per Table 1) is added to the blender and the formulation blended. This blended formulation is then roller compacted, milled, and then blended. The remainder or extragranular portion of the magnesium stearate (0.5%, per Table 1) is added and the final formulation is blended. Capsules are either mechanically filled or manually filled via the flood fill method.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

The study objective was to validate the use of Compound 1 besylate, an orally available, potent, small molecule inhibitor of Bruton's Tyrosine Kinase (Btk) in a mouse model of Systemic Lupus Erythematosis (SLE) in order to demonstrate its therapeutic utility to treat the human condition of SLE.

Compound 1 besylate was tested in the NZBxNZW Fl mouse SLE model. Treatment efficacy of Compound 1 besylate in this model was compared with cyclophosphamide, and a vehicle control. Principal end measures were autoantibodies against double stranded DNA (dsDNA) and protein in the urine.

Female NZBxNZW FI mice were housed in filter-top Nalgene cages, maintained on a 12-hour light/12-hour dark cycle with a room temperature of 70° F.+/−4° F. Mice were allowed to acclimate to the laboratory environment for approximately 5-7 days to ascertain their continued good health. Irradiated laboratory rodent chow and autoclaved water were supplied ad libitum.

Eighty NZBxNZW F1 female mice ˜12 weeks in age were obtained from Jackson Laboratory and were acclimatized for one week. After the acclimatization period was complete, each mouse that was successfully acclimatized was ear tagged and body weight recorded.

Urine analysis was initiated when the animals reached 13-14 weeks of age. Initially urine analysis was performed once in two weeks. When the animals reached 18-19 weeks of age, weekly urine analysis for proteinuria levels was performed using Albustix® Reagent strips for Urinalysis (Bayer, Inc). Results were scored on a 0-4 scale using the following criteria:

| Range (mg/dl) | Score |
|---|---|
| Negative or trace protein | 0 |
| ~30 | 1 (+) |
| ~100 | 2 (++) |
| ~300 | 3 (+++) |
| >2,000 | 4 (++++) |

An initial bleed was performed prior to treatment initiation and serum prepared from the blood sampled. Serum samples were stored at −80° C. Following treatment initiation, blood was collected once every two weeks during the in-life phase for subsequent serum preparation.

When the mice reached 19 weeks of age, all animals were evaluated with respect to their proteinuria scores. Based on that evaluation, seventy mice were selected and assigned to 6 groups of 10 mice each and one group of 5 mice. The mice were assigned such that the distributions of proteinuria scores among all groups were as uniform as possible.

Treatment was administered when the mice reached ˜20 weeks of age as follows:

| Animal | | Route of | Group |
|---|---|---|---|
| Group | Dosage | Administration | Size |
| 1 | Vehicle: BID for 12 weeks | PO | 10 |
| (Negative | | | |
| Control) | | | |
| 2 | Compound 1 besylate: | PO | 10 |
| | 50 mg/kg BID for 12 weeks | | |
| 3 | Compound 1 besylate: | PO | 10 |
| | 30 mg/kg QD for 12 weeks | | |
| 4 | Compound 1 besylate: | PO | 10 |
| | 100 mg/kg QD for 12 weeks | | |
| 5 | Compound 1 besylate: | — | 10 |
| | 0.16 mg/mL in drinking water | | |
| | for 12 weeks | | |
| 6 | Cyclophosphamide: | IP | 10 |
| (Positive | 35 mg/kg once a week | | |
| Control) | for 12 weeks | | |
| 7* | — | — | 5 |

*Animals harvested one day prior to dosing initiation

Body weights were taken at initial entry of animals into the study and at two week intervals until the mice reached ˜19 weeks of age. Thereafter, body weight was recorded weekly.

Proteinuria: Urine was tested once every two weeks when the animals reached ˜13 weeks of age. Urine analysis was preformed midweek at approximately the same time of the day. Weekly urine analysis commenced when the animals reached the age of ˜18 weeks. Proteinuria levels were evaluated using Albustix urine strips. Serum was collected via collection of blood from tail bleeds when animals reached 13 weeks in age, then prior to initiating the treatment and thereafter twice a month.

Clinical observations: Mice were observed daily for morbidity and ambulatory discomfort. Animals under distress were handled as per Aragen's SOP on ‘Care of laboratory animals’.

On the final day of treatment, 50% of the animals from each group were harvested 4 hours following the morning dosing and the remaining 50% of the animals were harvested 24 hours later. During harvest, the animal was placed under anesthesia and sacrificed by exsanguination followed by cervical dislocation. Plasma was extracted from 100 μL of whole blood and serum was extracted from the remaining volume of whole blood.

Kidneys were harvested and weighed. The left kidney from each animal was fresh frozen in liquid nitrogen and stored at −80° C. The right kidney was fixed in 10% NBF for 24 hours and then transferred to 70% ethanol and stored at room temperature.

The spleens were harvested from all animals, weighed and sliced in half. One half of the spleen was fixed in 10% NBF for 24 hours and then transferred to 70% ethanol and stored at room temperature. The other half of the spleen was placed in a microcentrifuge tube, snap frozen over liquid nitrogen and stored at −80° C.

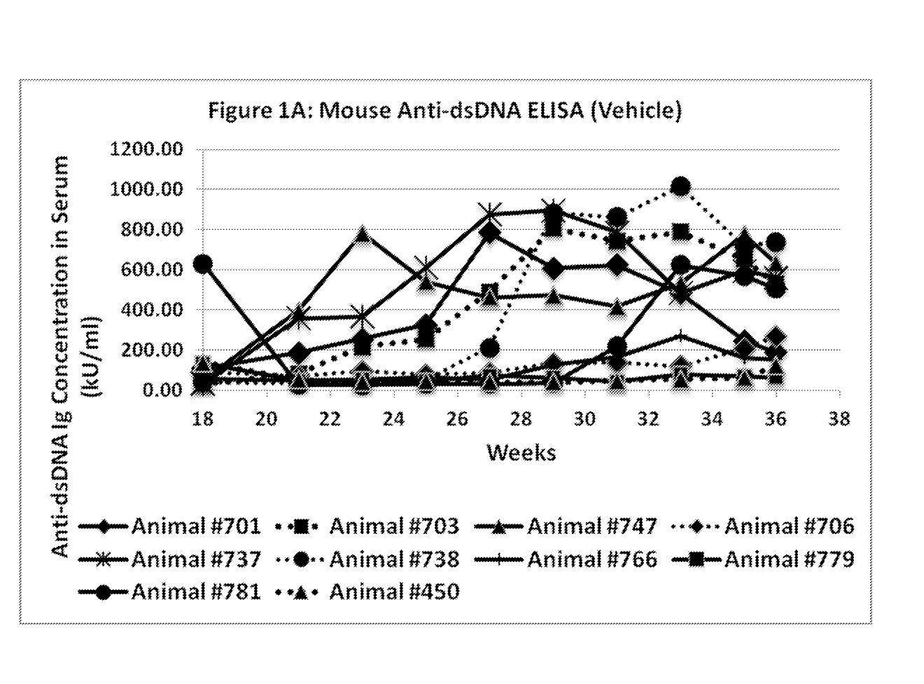

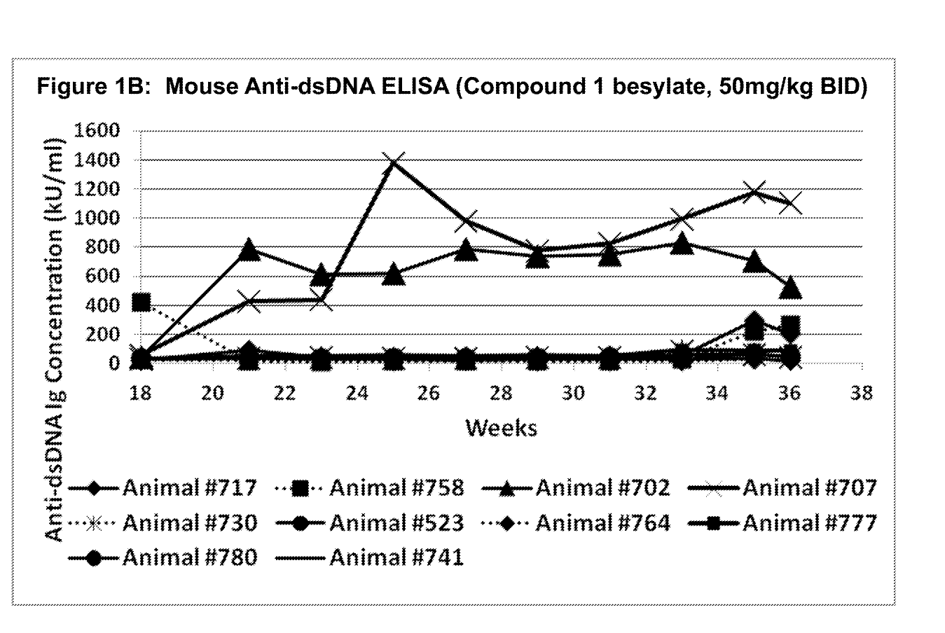

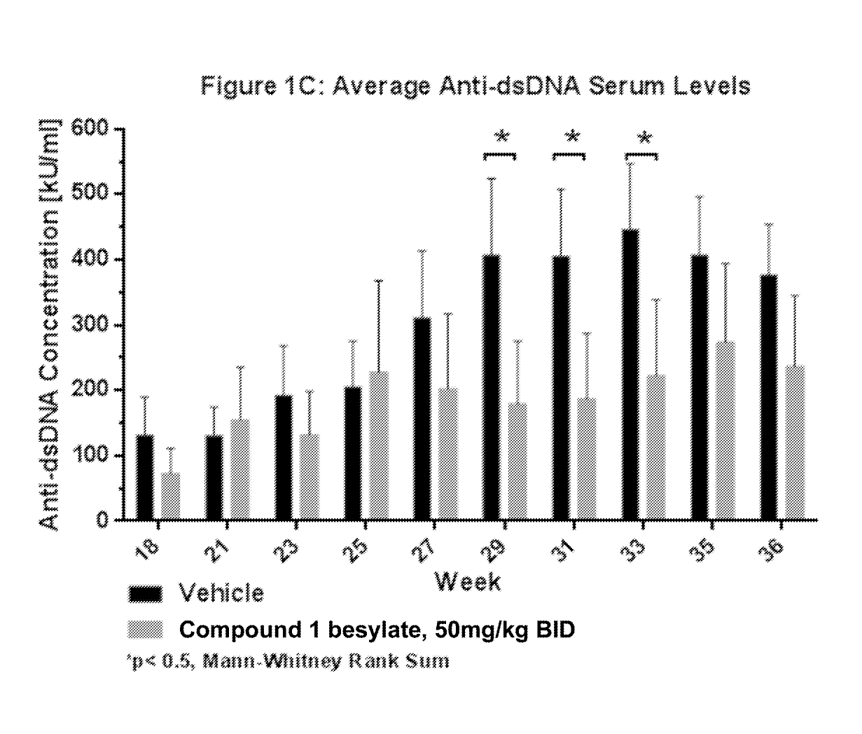

Results. Individual autoantibody development in NZBxNZW Fl mice over time is depicted in FIG. 1A. Serum samples were collected from vehicle treated mice at the times indicated and an anti-dsDNA quantitation by ELISA performed. During the course of measurement from 18 weeks of age to 36 weeks of age, six (6) out of ten (10) mice developed autoantibodies against dsDNA greater than 400 KU/ml during the course of disease development and eight out of ten developed dsDNA antibodies greater than 200 kU/ml.

Individual autoantibody development in NZBxNZW Fl mice treated with 50 mg/kg BID Compound 1 besylate is depicted in FIG. 1B. Two (2) of ten (10) treated mice developed dsDNA over 400 kU/ml antibodies compared with six (6) of ten (10) mice developing dsDNA over 400 kU/ml antibodies in vehicle-treated mice (FIG. 1A). Nearly complete suppression of anti-dsDNA development was observed in six (6) mice and two (2) had blunted anti-dsDNA responses at the end of the study (weeks 35 and 36).

The reduced autoantibody development in NZBxNZW F1 mice treated with 50 mg/kg BID Compound 1 besylate is depicted in FIG. 1C. Treatment with 50 mg/kg BID Compound 1 besylate resulted in reduced anti-dsDNA autoantibody development from weeks 29 through 33.

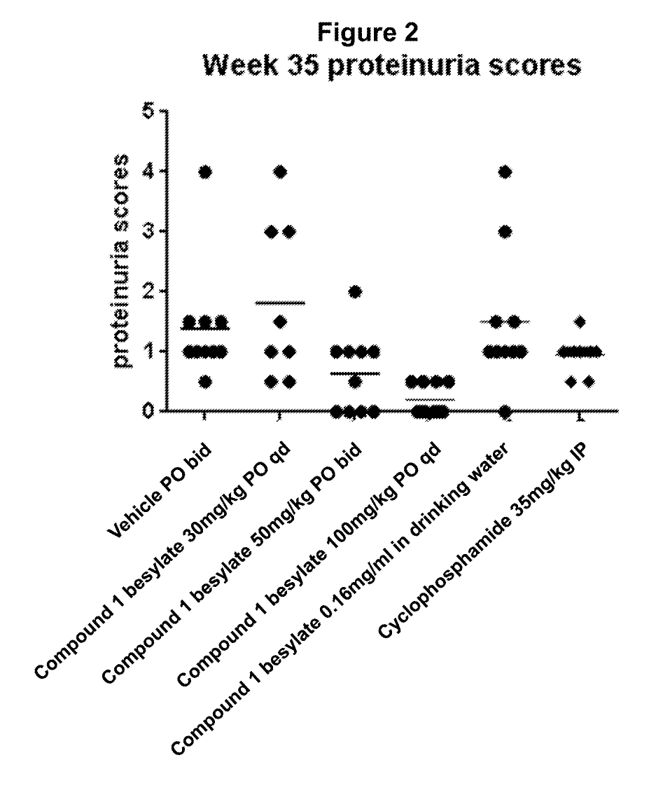

FIG. 2 depicts the reduced urine protein levels in NZBxNZW F1 mice treated with Compound 1 besylate. Mice treated with vehicle, Compound 1 besylate, or cyclophosphamide at the indicated doses are shown. Mice treated with Compound 1 besylate at 50 mg/kg BID and 100 mg/kg QD showed reduced protein levels measured in the urine relative to vehicle.

Both the suppression of anti-dsDNA autoantibody development in treated mice and the reduction of urine protein levels in treated mice are suggestive of an effect of Compound 1 besylate in this animal model of lupus. Compound 1 besylate exhibited a dose-associated trend of reduced urine protein despite the limitation of the moderate urine protein kidney pathology having developed in the NZBxNZW F1 SLE mouse model shown in FIG. 1. Without wishing to be bound to theory, the clinical reduction of autoantibodies and/or B-cells producing them are features useful in antiautoimmune compounds; Compound 1 (or pharmaceutically acceptable salts thereof) may be useful to treat the autoreactive antibodies generated in lupus and other autoimmune disorders. For example, FIGS. 1A-1C show a trend of suppression of anti-dsDNA autoantibody development in treated mice. Further, without wishing to be bound to theory, downstream benefits are thought to be reduction in tissue inflammation and the pathologies that develop from lupus and other autoimmune disorders. An example of the ability of Compound 1, or pharmaceutically acceptable salts thereof, to reduce kidney inflammation and the subsequent damage is evidenced by reduced protein in the urine in the NZBxNZW F1 mouse SLE model (FIG. 2).

We claim:

1. A method of preventing, treating, stabilizing or lessening the severity or progression of an autoimmune disorder, the method comprising administering to a patient in need thereof a pharmaceutically acceptable composition comprising a therapeutically effective amount of N-(3-(5-fluoro-2-(4-(2-methoxyethoxy)phenylamino)pyrimidin-4-ylamino)phenyl)acrylamide (1): 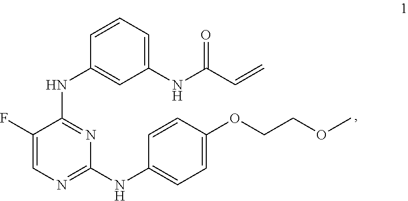 [SMILES: C=CC(=O)Nc1cccc(Nc2nc(Nc3ccc(OCCOC)cc3)ncc2F)c1]
or a pharmaceutically acceptable salt thereof.

2. The method according to claim 1, wherein the therapeutically effective amount of Compound 1 is about 125 mg BID to about 750 mg BID.

3. The method according to claim 1, wherein the therapeutically effective amount of Compound 1 is about 125 mg to about 375 mg.

4. The method according to any of claims 1-3, wherein the autoimmune disorder is selected from lupus, Graves' disease, Hashimoto's thyroiditis, myasthenia gravis, mixed connective tissue disease, celiac disease, inflammatory myopathy, diabetes mellitus type 1, and Lambert-Eaton myasthenic syndrome.

5. The method according to claim 4, wherein the autoimmune disorder is lupus.

6. The method according to claim 5, wherein the lupus is systemic lupus erythematosis (SLE).

7. The method according to claim 5, wherein the lupus is drug-induced lupus erythematosus.

8. The method according to any of claims 1-7, wherein the pharmaceutically acceptable composition is formulated as an oral dosage form.

9. The method according to claim 1, wherein the pharmaceutically acceptable composition is administered twice a day.

10. The method according to any of claims 1-9, wherein the pharmaceutically acceptable composition is administered for at least one 28-day cycle.

11. The method according to any of claims 1-10, wherein Compound 1 is administered as a salt.

12. The method according to claim 11, wherein the salt is a benzenesulfonic acid salt.

13. The method according to claim 12, wherein the composition comprises from about 10% to about 50% N-(3 -(5 -fluoro-2-(4-(2-methoxyethoxy)phenylamino)pyrimidin-4-ylamino)phenyl)acrylamide besylate.

14. The method according to claim 13, wherein the composition comprises about 42% N-(3-(5-fluoro-2-(4-(2-methoxyethoxy)phenylamino)pyrimidin-4-ylamino)phenyl)acrylamide besylate.

15. The method according to claim 13 or claim 14, wherein the composition comprises from about 5% to about 15% by weight of wetting agent.

16. The method according to claim 15, wherein the composition comprises about 10% by weight of wetting agent.

17. The method according to claim 15 or 16, wherein the wetting agent is selected from poloxamer, polyoxyethylene ethers, polyoxyethylene sorbitan fatty acid esters polyoxyethylene fatty acid esters, polyethylene glycol fatty acid esters, polyoxyethylene hydrogenated castor oil, polyoxyethylene alkyl ether, polysorbates, cetyl alcohol, glycerol fatty acid esters, polyoxymethylene stearate, sodium lauryl sulfate, sorbitan fatty acid esters, sucrose fatty acid esters, benzalkonium chloride, polyethoxylated castor oil, and docusate sodium.

18. The method according to claim 17, wherein the wetting agent is a poloxamer.

19. The method according to claim 18, wherein the poloxamer is poloxamer 407.

20. The method according to any of claims 1-19, wherein the therapeutically effective amount is about 125 mg BID.

21. The method according to any of claims 1-19, wherein the therapeutically effective amount is about 250 mg BID.
